(19) United States
(12) Patent Application Publication
(10) Pub. No.: US 2016/0213569 A1
(43) Pub. Date: Jul. 28, 2016

(54) PHARMACEUTICAL COMPOSITIONS OF RANOLAZINE AND DRONEDARONE

(71) Applicant: Gilead Sciences, Inc., Foster City, CA (US)

(72) Inventors: Michael Gerber, Oakland, CA (US); Neal Huang, San Francisco, CA (US); Joanna M. Koziara, Foster City, CA (US); Feng Zhang, Pflugerville, TX (US)

(21) Appl. No.: 15/092,126

(22) Filed: Apr. 6, 2016

Related U.S. Application Data
(62) Division of application No. 14/445,986, filed on Jul. 29, 2014.
(60) Provisional application No. 61/861,862, filed on Aug. 2, 2013.

(51) Int. Cl. A61J 3/10; A61K 9/24; A61K 9/20; A61K 31/495; A61K 31/343

(52) U.S. Cl. CPC A61J 3/10; A61K 31/495; A61K 31/343; A61K 9/2095; A61K 9/209

(57) ABSTRACT

The present disclosure relates to a solid composition comprising ranolazine and a spray-dried phosphoric acid salt of dronedarone in a bilayer tablet.

### CROSS REFERENCE TO RELATED APPLICATIONS

This application claims the benefit under 35 U.S.C. §119(e) to U.S. Provisional Application Ser. No. 61/861,862, filed on Aug. 2, 2013, the entirety of which is incorporated herein by reference.

### FIELD OF THE INVENTION

The present invention relates to a solid pharmaceutical composition comprising of ranolazine and dronedarone and methods for treating and/or preventing atrial fibrillation and/or atrial flutter.

### BACKGROUND

Atrial fibrillation (AF) is the most prevalent arrhythmia, the incidence of which increases with age. It is estimated that 8% of all people over the age of 80 experience this type of abnormal heart rhythm and AF accounts for one-third of hospital admissions for cardiac rhythm disturbances. Over 2.2 million people are believed to have AF in the Unites States alone. Fuster, et al Circulation 2006 114 (7): e257-354. Although atrial fibrillation is often asymptomatic it may cause palpitations or chest pain. Prolonged atrial fibrillation often results in the development of congestive heart failure and/or stroke. Heart failure develops as the heart attempts to compensate for the reduced cardiac efficiency while stroke may occur when thrombi form in the atria, pass into the blood stream and lodge in the brain. Pulmonary emboli may also develop in this manner.

In the U.S., the antiarrhythmic drug Multaq® (dronedarone HCl) is indicated to reduce the risk of cardiovascular hospitalization in patients with paroxysmal or persistent atrial fibrillation (AF) or atrial flutter (AFL) with a recent episode of AF or AFL and associated cardiovascular risk factors (i.e. age >70, hypertension, diabetes, prior cerebrovascular accident, left ventricular ejection fraction (LVEF)<40%, who are in a sinus rhythm or who will be cardioverted. U.S. Pat. No. 5,223,510 discloses alkylaminoalkyl derivatives of benzofuran, benzothiophene, indole and indolizine, process for their preparation and compositions containing them. PCT International Publication WO 2011/1.35581 describes pharmaceutical compositions of dronedarone.

Ranexa® (ranolazine sustained release formulation) is indicated in the U.S. for the treatment of chronic angina. Sustained release formulations of ranolazine are described in, for example, U.S. Pat. No. 6,503,911.

PCT International publication WO 2011/084733A1 discloses that the use of a combination of dronedarone HCl and ranolazine has synergism resulting in potent electrophysiologic actions leading to marked suppression of atrial arrhythmias among other cardiac conditions. For example, the use of dronedarone and ranolazine in combination showed synergism in reducing AV nodal conduction and ventricular tachyarrhythmia.

The above disclosures notwithstanding, there remains a need to discover new formulations of a solid combination of ranolazine and dronedarone for the treatment of arrhythmias, particularly atrial fibrillation or atrial flutter.

### SUMMARY OF THE DISCLOSURE

The present disclosure provides a bilayer tablet comprising ranolazine and one or more pharmaceutically acceptable excipients in a first layer, and a spray-dried phosphoric acid salt formulation of dronedarone further comprising HPMC E3 or HPMC E5 and one or more pharmaceutically acceptable excipients in a second layer.

The present disclosure provides a bilayer tablet comprising ranolazine and one or more pharmaceutically acceptable excipients in a first layer, and a stable solid spray-dried phosphoric acid salt formulation of dronedarone further comprising HPMC E3 or HPMC E5 and one or more pharmaceutically acceptable excipients in a second layer.

The present disclosure provides a process for making a stable spray-dried formulation of the phosphoric acid salt of dronedarone.

The present disclosure provides a process for making a stable spray-dried formulation of the phosphoric acid salt of dronedarone suitable for forming a solid bilayer tablet comprising dronedarone and ranolazine.

The present disclosure provides a process for making a bilayer tablet comprising ranolazine and one or more pharmaceutically acceptable excipients in a first layer, and a stable solid spray-dried phosphoric acid salt formulation of dronedarone and one or more pharmaceutically acceptable excipients in a second layer.

PCT International Publication WO 2012/032545 published Mar. 15, 2012 discloses generally, the formation of salts including phosphate salts of dronedarone. Further, WO 2012/032545 discloses spray-drying of salts and further discloses that “any known form of pharmaceutically acceptable acid addition salt of dronedarone and the filtered cake that is obtained as an end result of the reaction or reaction mass comprising pharmaceutically acceptable acid addition salts of dronedarone or solution comprising pharmaceutically acceptable acid addition salts of dronedarone can be used for the preparation of feed stock (for spray-drying)” (emphasis added). However, WO 2012/032545 does not provide an enabling disclosure for the preparation of phosphate salt (phosphoric acid salt) of dronedarone. Applicants' efforts to prepare phosphoric acid salt of dronedarone invariably led to a substance which is a yellowish, congealed, and/or sticky mass which is also unstable at 45° C. and 75% relative humidity (RH). Furthermore, such congealed sticky product was unsuitable for forming a solid composition comprising ranolazine and the phosphoric acid salt of dronedarone. Applicants have unexpectedly and surprisingly discovered that a particular polymer (hydroxypropyl methylcellulose E5 (HPMC E5) or hydroxypropyl methylcellulose E3 (HPMC E3)) is necessary for the formation of a stable solid phosphoric acid salt of dronedarone. Applicants' disclosure herein enables preparation of a stable spray-dried phosphoric acid salt formulation of dronedarone suitable for forming a solid bilayer tablet with ranolazine. Thus, one aspect of the present disclosure is a process for the manufacture of a spray-dried formulation of phosphoric acid salt of dronedarone suitable for tablet formation.

Another aspect of the present disclosure is the use of the spray-dried formulation of phosphoric acid salt of dronedarone disclosed herein in combination with ranolazine to form a bilayer tablet.

Another aspect of the present disclosure is the use of the spray-dried formulation of phosphoric acid salt of dronedarone (disclosed herein) in combination with ranolazine in a bilayer tablet wherein the ranolazine is present as a sustained release formulation.

Yet another aspect of the present disclosure is a process for making a stable solid spray-dried phosphoric acid salt formulation of dronedarone comprising the steps of:

a. dissolving the base form of dronedarone in a solution of phosphoric acid to form a dronedarone solution;

b. optionally adjusting the pH of the dronedarone solution from step (a) to about 4.0 with additional phosphoric acid as necessary;

c. adding HPMC E3 or HPMC E5 to the dronedarone solution from step (b);

d. spray drying the dronedarone solution from step (c) to achieve a solid spray-dried phosphoric acid salt formulation of dronedarone; and

e. optionally drying the solid spray-dried phosphoric acid salt formulation of dronedarone.

Yet another aspect of the present disclosure is a process for making a stable solid spray-dried phosphoric acid salt formulation of dronedarone comprising the steps of:

a. dissolving the base form of dronedarone in a solution of phosphoric acid to form a dronedarone solution;

b. adjusting the pH of the dronedarone solution from step (a) to about 4.0 with additional phosphoric acid as necessary;

c. adding HPMC E3 or HPMC E5 to the dronedarone solution from step (b);

d. spray drying the dronedarone solution from step (c) to achieve a solid spray-dried phosphoric acid salt formulation of dronedarone; and

e. optionally drying the solid spray-dried phosphoric acid salt formulation of dronedarone.

Another aspect of the present disclosure is a process for making a bilayer tablet comprising ranolazine in a first layer and stable solid spray-dried phosphoric acid salt formulation of dronedarone in a second layer further comprising the steps of:

a. providing a powder blend of stable solid spray-dried phosphoric acid salt formulation of dronedarone with suitable excipients;

b. processing the powder blend from step (a) into granules with suitable flow and compression properties;

c. providing a powder blend of ranolazine with suitable excipients;

d. processing the ranolazine from step (c) with suitable excipients into granules with suitable flow and compression properties; and

e. forming a bilayer tablet by compressing dronedarone granules from step (b) and the ranolazine granules from step (d) using a bilayer tablet press, wherein the ranolazine granules are in a first layer and the dronedarone granules are in a second layer.

Another aspect of the present disclosure is a process for making a bilayer tablet comprising ranolazine in a first layer and stable solid spray-dried phosphoric acid salt formulation of dronedarone in a second layer further comprising the steps of:

a. providing a powder blend of stable solid spray-dried phosphoric acid salt formulation of dronedarone with suitable excipients;

b. optionally processing the powder blend from step (a) into granules with suitable flow and compression properties;

c. providing a powder blend of ranolazine with suitable excipients;

d. processing the ranolazine from step (c) with suitable excipients into granules with suitable flow and compression properties; and

e. forming a bilayer tablet by compressing dronedarone granules or powder blend from step (b) and the ranolazine granules from step (d) using a bilayer tablet press, wherein the ranolazine granules are in a first layer and the dronedarone granules are in a second layer.

In another aspect, the present disclosure provides a solid pharmaceutical composition comprising ranolazine, a spray-dried phosphoric acid salt formulation of dronedarone and a pharmaceutically acceptable carrier(s) in a fixed dose combination wherein the spray-dried phosphoric acid salt formulation of dronedarone is formed by the admixture of HPMC E5 or HPMC E3, dronedarone, and phosphoric acid solution and spray-drying the resulting solution or mixture.

In another aspect, the present disclosure provides a bilayer tablet comprising ranolazine and one or more pharmaceutically acceptable excipients in a first layer, and a spray-dried phosphoric acid salt formulation of dronedarone and one or more pharmaceutically acceptable excipients in a second layer wherein the first layer comprises a sustained release formulation of ranolazine and wherein the second layer further comprises HPMC E3 or HPMC E5.

In yet another embodiment, present disclosure provides a bilayer tablet comprising ranolazine and one or more pharmaceutically acceptable excipients in a first layer, and a spray-dried phosphoric acid salt formulation of dronedarone and one or more pharmaceutically acceptable excipients in a second layer wherein the first layer comprises a sustained release formulation of ranolazine and wherein the second layer further comprises HPMC E3 or HPMC E5 in ratio of dronedarone to HPMC E3 or HPMC E5 polymer is from about 0.5:1 to about 15:1, or from about 1:1 to about 10:1, or from about 1:1 to about 6:1 or from about 1:1 to about 2:1.

In another aspect the present disclosure provides a pharmaceutical composition consisting essentially of sustained release ranolazine and spray-dried phosphoric acid salt formulation of dronedarone wherein the spray-dried phosphoric acid salt formulation of dronedarone further comprises HPMC E3 or HPMC E5.

In a preferred embodiment, the present disclosure provides a solid pharmaceutical composition comprising sustained release formulation of ranolazine, spray-dried phosphoric acid salt formulation of dronedarone, and pharmaceutically acceptable carrier(s).

### DETAILED DESCRIPTION OF THE DISCLOSURE

### Definitions and General Parameters

As used in the present specification, the following words and phrases are generally intended to have the meanings as set forth below, except to the extent that the context in which they are used indicates otherwise.

It is to be noted that as used herein and in the claims, the singular forms “a,” “an” and “the” include plural referents unless the context clearly dictates otherwise. Thus, for example, reference to “a pharmaceutically acceptable carrier” in a composition includes two or more pharmaceutically acceptable carriers, and so forth.

The term “as necessary” as used herein in connection with adjusting the pH of the phosphoric acid solution of dronedarone means that the practitioner will, depending on the initial pH of the solution add more phosphoric acid solution to achieve a pH of about 4.0. Where the pH is already about 4.0, there will be no need to add more phosphoric acid solution. As used herein the pH values are generally are measured at room temperature typically about 20-25 degrees Celsius.

As used herein “HPMC E3” and “HPMC E5” refer respectively to specific grades of hydroxypropyl methyl cellulose of substitution type E as defined by Dow Chemical Company. Both materials can be sourced from Dow Chemical Company. Hydroxypropyl cellulose is referred to as “Hypromellose” in United States Pharmacopeia. Substitution type E is referred to as substitution 2910 in United State Pharmacopeia. Further, HPMC E3 is characterized as having a viscosity of 2.4-3.6 in a 2% solution cps and E5 is characterized as having a viscosity of 4.0-6.0 in a 2% solution cps.

“Dronedarone” or “Drop” is described in U.S. Pat. No. 5,223,510. Dronedarone refers to the chemical compound, N-{2-butyl-3-[4-(3-dibutylaminopropoxy)benzoyl]benzofuran-5-yl. The base form of dronedarone (dronedarone base) has the following chemical formula:

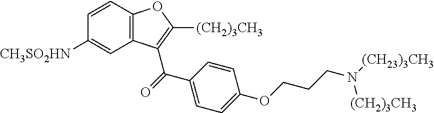

The phosphoric acid salt of dronedarone has the following chemical formula:

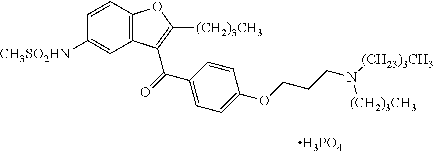

“Ranolazine” is described in U.S. Pat. No. 4,567,264. It refers to the chemical compound (±)-N-(2,6-dimethylphenyl)-4-[2-hydroxy-3-(2-methoxyphenoxy)-propyl]-1-piperazineacetamide. In its dihydrochloride salt form, ranolazine is represented by the formula:

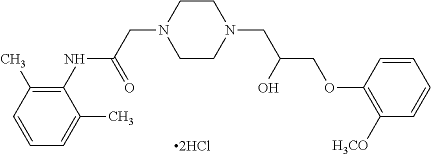 [SMILES: COc1ccccc1OCC(O)CN1CCN(CC(=O)Nc2c(C)cccc2C)CC1]

The term “powder blend” refers to the result of mixing, blending or milling and subsequent blending or mixing of the non-uniform powder or particles of a compound to achieve uniformity in particle size and/or flow properties. Thus the term, “preparing a powder blend” refers to the act of attaining uniformity in particle size and/or flow properties by blending i.e. mixing, milling, etc. One of skill in the art is aware of processes for preparing a powder blend.

The term “providing a powder blend,” refers to the act of using a powder blend prepared as above.

The term “solid dispersion tablets” or “dronedarone dispersion tablets” as used herein refer to the tablets produced via a process to prepare spray-dried phosphoric acid salt of dronedarone as described herein.

The term “spray-dried phosphoric acid salt formulation of dronedarone” refers to the product of the spray-drying process described herein, i.e. the result of spray-drying the admixture of dronedarone, phosphoric acid, and HPMC E3 or HPMC E5 with or without a carrier or additional excipients.

The term “therapeutically effective amount” refers to that amount of a compound, such as ranolazine or dronedarone or combination thereof, that is sufficient to effect treatment, as defined below, when the subject compound or combination of compounds is administered to a human patient in need of thereof. The therapeutically effective amount may vary depending on the severity of the patient's disease state, the age, physical condition, existence of other disease states, and nutritional status of the patient. Additionally, other medication(s) the patient may be receiving may affect the determination of the therapeutically effective amount of the therapeutic agent to be administered. In some embodiments, the term “therapeutically effective amount” refers to a synergistically effective amount of each ingredient in a combination.

As used herein, the term “stable solid” used in reference to the stability of a spray-dried phosphoric acid salt formulation of dronedarone, implies a solid or solid formulation that is stable at 25° C. at 60% RH (relative humidity) for at least five months. A stable solid is stable under stressed conditions such as 40° C. at 75% RH open conditions for at least 5 months. Additionally, a stable solid is stable in suitable packaging when stored at 40° C. at 75% for at least 5 months.

As used herein the term “synergistic” means that the therapeutic effect of dronedarone when administered in combination with ranolazine (or vice-versa) is greater than the predicted additive therapeutic effects of dronedarone and ranolazine when administered one without the other. The term “synergistically therapeutic amount” may refer to a less than standard therapeutic amount of one or both drugs, meaning that the amount required for the desired effect is lower than when either of the drugs is used alone. A synergistically therapeutic amount also includes when one drug is given at a standard therapeutic dose and another drug is administered in a less than standard therapeutic dose. For example, ranolazine could be given in a therapeutic dose and dronedarone could be given in a less than standard therapeutic dose to provide a synergistic result.

The term “treatment” or “treating” refers to administration of a medicament or drug composition according to the present disclosure to a human for the purpose of: 1) preventing or protecting against the disease or condition, i.e. causing the clinical symptoms not to develop; 2) inhibiting the disease or condition, i.e. arresting or suppressing the development of clinical symptoms; and/or 3) relieving the disease or condition i.e. causing the regression of clinical symptoms.

As used herein, a “pharmaceutically acceptable carrier” includes any and all diluents, excipients, solvents, dispersion media, coatings, antibacterial and antifungal agents, isotonic and absorption delaying agents and the like that are found suitable for the purpose of formulating the combined dosage form as disclosed herein and consistent with the invention or object of the invention as disclosed herein. The use of such media or agents for pharmaceutically active substances is well known in the art. Except where a conventional media or agent is incompatible with the active ingredient or excluded by specific limitation of the disclosure herein, its use in the therapeutic compositions herein is contemplated. One of skill in the art in the pharmaceutical sciences is aware of pharmaceutically acceptable carriers and their uses in drug formulation.

As used herein “immediate release” (“IR”) refers to formulations or dosage units that rapidly dissolve in vitro and are intended to be completely dissolved and/or absorbed in the stomach or upper gastrointestinal tract within 30 minutes of administration.

As used herein, “sustained release” (“SR”) refers to formulations or dosage units that are slowly and continuously dissolved and absorbed in the stomach and gastrointestinal tract over a period of about six hours or more. Preferred sustained release formulations of ranolazine are those exhibiting plasma concentrations of ranolazine suitable for no more than twice daily administration with two or less tablets per dosing. Suitable plasma ranolazine concentrations are known to one of skill in the art and are disclosed in, for example, U.S. Pat. Nos. 6,503,911, 6,617,328, 6,303,607, 6,369,062, 6,525,057, 6,562,826, 6,620,814, 6852724, and 6864258 incorporated herein by reference. A preferred embodiment of the present invention is the use of a sustained release formulation of ranolazine. However, it is contemplated that an immediate release formulation of ranolazine may also be used in the practice of the invention.

### Methods

Prior to the present disclosure, a stable solid formulation comprising the phosphoric acid salt of dronedarone had not been disclosed or otherwise described. Applicants' initial efforts to prepare stable solid forms of the phosphoric acid salt of dronedarone were unsuccessful, resulting invariably in a congealed, sticky, yellowish mass. Applicants' research resulted in the discovery that adding the polymer HPMC E5 or HPMC E3 to a solution of the phosphoric acid salt of dronedarone prior to spray drying, results in a stable solid spray-dried phosphoric acid salt formulation of dronedarone. Surprisingly, Applicants also observed that HPMC E5 and HPMC E3 each produced stable spray-dried salts of dronedarone only with phosphoric acid compared to use with other counter ions tested. While not being bound by theory, Applicants hypothesize that a complex is formed between HPMC E3 or HPMC E5 and the phosphoric acid salt of dronedarone that enables conversion of the hitherto unstable salt to a stable salt upon spray-drying.

Accordingly, the present disclosure provides a stable solid spray-dried formulation of the phosphoric acid salt of dronedarone. The spray-dried phosphoric acid salt formulation of dronedarone as described herein, provides improved stability and manufacturability of, for example, tablets for oral administration comprising ranolazine and the spray-dried phosphoric acid salt formulation of dronedarone. According to the present disclosure, the solid spray-dried phosphoric acid salt formulation of dronedarone is used to form a stable solid fixed dose combination of ranolazine and the phosphoric acid salt of dronedarone.

In one embodiment, the disclosure provides a process for making a stable solid spray-dried phosphoric acid salt formulation of dronedarone comprising the steps of:

a. dissolving the base form of dronedarone in a solution of phosphoric acid to form a dronedarone solution;

b. optionally adjusting the pH of the dronedarone solution from step (a) to about 4.0 with additional phosphoric acid as necessary;

c. adding HPMC E3 or HPMC E5 to the dronedarone solution from step (b);

d. spray drying the dronedarone solution from step (c) to provide a solid comprising spray-dried phosphoric acid salt formulation of dronedarone; and

e. optionally drying the solid spray-died phosphoric acid salt formulation of dronedarone.

In yet another embodiment, the disclosure provides a process for making a stable solid spray-dried phosphoric acid salt formulation of dronedarone comprising the steps of:

a. dissolving the base form of dronedarone in a solution of 1:1 molar equivalent of phosphoric acid (based on dronedarone base) to form a dronedarone solution;

b. adding HPMC E3 or HPMC E5 or solution thereof to the dronedarone solution from step (a);

c. spray drying the dronedarone solution from step (b) to achieve a solid spray-dried dronedarone phosphoric acid salt formulation; and

d. optionally drying the solid spray-dried phosphoric acid salt formulation of dronedarone.

Also provided is a process for making a bilayer tablet comprising ranolazine in a first layer and stable solid spray-dried phosphoric acid salt formulation of dronedarone in a second layer further comprising the steps of:

a. providing a powder blend of stable solid spray-dried phosphoric acid salt formulation of dronedarone with suitable excipients;

b. optionally processing the powder blend from step (a) into granules with suitable flow and compression properties;

c. providing a powder blend of ranolazine with suitable excipients;

d. processing the powder blend from step (c) with suitable excipients into granules with suitable flow and compression properties; and

forming a bilayer tablet by compressing granules from step (b) or powder blend from step (a) and the granules from step (d) using a bilayer tablet press, wherein the granules from step (b) are in a first layer and the granules from step (b) or powder blend from step (a) are in a second layer.

In yet another embodiment, the disclosure provides a process for making a stable solid spray-dried phosphoric acid salt formulation of dronedarone comprising the steps of:

a. dissolving HPMC E3 or HPMC E5 and the base form of dronedarone in a suitable solvent or solvent mixture that contains 1 molar equivalent of phosphoric acid (based on dronedarone base) to form a dronedarone solution;

b. spray drying the dronedarone solution from step (a) to achieve a solid spray-dried dronedarone phosphoric acid salt formulation; and

optionally drying the solid spray-dried phosphoric acid salt formulation of dronedarone.

Also provided is a process for making a bilayer tablet comprising ranolazine in a first layer and spray-dried phosphoric acid salt formulation of dronedarone in a second layer further comprising the steps of:

a. providing a powder blend of stable solid spray-dried phosphoric acid salt formulation of dronedarone with suitable excipients;

b. processing the powder blend from step (a) into granules with suitable flow and compression properties;

c. providing a powder blend of ranolazine with suitable excipients;

d. processing the powder blend from step (c) with suitable excipients into granules with suitable flow and compression properties; and

e. forming a bilayer tablet by compressing granules from step (b) and the granules from step (d) using a bilayer tablet press, wherein the granules from step (b) are in a first layer and the granules from step (d) are in a second layer.

In another embodiment, the disclosure provides a process for making a bilayer tablet comprising ranolazine and spray-dried phosphoric acid salt formulation of dronedarone further comprising the steps of:

a. providing granules of spray-dried phosphoric acid salt formulation of dronedarone;

b. providing granules of ranolazine

c. forming a bilayer tablet by compressing dronedarone granules from step (a) and the ranolazine granules from step (b) using a bilayer tablet press, wherein the dronedarone and ranolazine granules are in separate layers.

In yet another embodiment, the disclosure provides a process for making a bilayer tablet comprising sustained release (SR) formulation of ranolazine and spray-dried phosphoric acid salt formulation of dronedarone further comprising the steps of:

a. providing granules of spray-dried phosphoric acid salt formulation of dronedarone;

b. providing granules of sustained release formulation of ranolazine

c. forming a bilayer tablet by compressing dronedarone granules from step (a) and the ranolazine granules from step (b) using a bilayer tablet press, wherein the dronedarone and ranolazine granules are in separate layers.

To prepare a feed solution of dronedarone, dronedarone base is dispersed in diluted phosphoric acid solution of about 1 to 2% w/w and gradually dissolved (optionally with mixing) as a result of its reaction with phosphoric acid. 95% of the theoretical amount of phosphoric acid is initially charged to prepare the feed solution. After the dronedarone base has dissolved, the remaining phosphoric acid solution is added as necessary to adjust the pH of the solution of dronedarone to about 4.0. Alternatively, stoichiometrically equivalent (1:1 molar equivalent) amount of phosphoric acid (based on dronedarone base) is charged (added) to the solution of dronedarone without additional adjustment of pH. Separately, a polymer solution is prepared by dispersing HPMC E3 LV or HPMC E5 LV powder in water and gradually dissolved with gentle mixing. The phosphoric acid solution of dronedarone and the polymer solution are mixed to prepare the feed solution for spray drying. As would be understood by one of ordinary skill in art, the polymer may be added directly to a solution of dronedarone in phosphoric acid or the polymer may be dissolved or dispersed in a solvent or co-solvent system, and the solution or dispersion added to a solution of dronedarone optionally with mixing. The resulting solution of dronedarone, phosphoric acid and HPMC E3 or E5 is then spray-dried. Thus, it is also an embodiment of the present disclosure to change the order of operation e.g. the order of addition of dronedarone solution in phosphoric acid (slight subcess to slight excess as disclosed herein) to the solution of HPMC E3 or HPMC E5 polymer or solution thereof. For example, one embodiment involves dissolving HPMC E3 or HPMC E5 and the base form of dronedarone in a solution of about 1 molar equivalent of phosphoric acid Based on dronedarone base) to form a dronedarone solution. Thus it is within the ambit of the disclosure to change order of steps or perform certain steps simultaneously or combine certain steps except the last step of an embodiment of the disclosure, all unless specified otherwise. Solvent systems useful for the practice of the disclosure include those listed in examples herein and comparable systems known to one of skill in the art. Example co-solvent systems are ethanol/water and acetone/water mixtures with a range of compositions from 1:99 (co-solvent: water by weight) to 90:10.

Spray-drying apparati and configurations thereof, are known to one of skill in the art. The spray-dryer uses an atomizer or spray nozzles to disperse the feed solution into a spray of controlled droplet size into a drying chamber. Inside the drying chamber, heated air or nitrogen can be used as the drying medium. The hot drying medium can be passed as a co-current or counter-current flow to the droplet direction. Inside the drying chamber, water and/or solvent evaporates rapidly from the surface of the droplets in the initial stage, which is followed by a falling drying rate period where the drying is controlled by diffusion of water and/or solvent to the surface of the particles. Separation of dried powder from drying gas is carried out using a cyclone or bag filter. In a closed loop configuration, drying gas is recycled back into the drying chamber after water and/or solvent is removed using a condenser. Upon the completion of the spray drying, the powder collected may undergo secondary drying to further reduce the water and/or solvent content.

One aspect of the disclosure is to provide a formulation of a bilayer tablet wherein one layer comprises ranolazine, preferably as the sustained release formulation, and the other layer comprises dronedarone as the spray-dried phosphoric acid salt formulation. The preferred amounts of active ingredients are as described herein. For preparing solid composition of bilayer tablets according to the present disclosure, the principal active ingredients, ranolazine (preferably sustained release form) and spray-dried phosphoric acid salt formulation of dronedarone are separately mixed with excipients prior to granulation and compression. Alternatively, the solid composition of bilayer tablets according to the present disclosure, ranolazine (preferably sustained release form) is mixed with excipients and granulated, while the spray-dried phosphoric acid salt formulation is separately mixed with excipients prior to compression. One of skill in the art is aware of methods, non-essential reagents and apparati for forming bilayer tablets.

In one embodiment, a first ingredient e.g. sustained release ranolazine formulation in the desired amount is compressed as the first layer into a loose compact with a low compression force in a rotary tablet press. The spray-dried phosphoric acid salt formulation of dronedarone is then filled into the die as the second layer (or vice versa). Both drug layers are then compressed again with a compression force sufficient to produce a bilayer tablet with acceptable hardness, friability and dissolution properties known to one of skill in the art. Further, the compressed tablets may be coated or otherwise compounded to provide a dosage form affording the advantage of prolonged action, or to protect from the acid conditions of the stomach, or to mask taste, or to make of a desired taste. For example, an embodiment of a bilayer tablet comprising an inner dosage element (drug) and an outer dosage element, the latter being in the form of an envelope over the former is also contemplated as within the ambit of the present disclosure. Ranolazine and the phosphoric acid salt formulation of dronedarone may be separated by an enteric layer that serves to resist disintegration in the stomach and permit the inner element to pass intact into the duodenum or to be delayed in release. A variety of materials can be used for such enteric layers or coatings including a number of polymeric acids and mixtures of polymeric acids with such materials as shellac, cetyl alcohol, and cellulose acetate. While the formation of bilayer tablets is preferred, one of skill in the art is aware that the scope of the present invention encompasses the formation of bilayer capsules or pills comprising for example, ranolazine formulation on the one side and a spray-dried phosphoric acid salt formulation of dronedarone on the other side.

### Dosing

It is contemplated that dronedarone in its spray-dried phosphoric acid salt formulation as described herein and ranolazine as the sustained release formulation will be administered in a fixed dose combination e.g. a bilayer tablet, in a therapeutically effective amount of each. In one embodiment of the bilayer tablet, the dronedarone is present in a synergistically effective dose and ranolazine is present in a standard therapeutically effective dose. In other embodiment, ranolazine is present in a less than standard therapeutic dose and dronedarone is present in a standard therapeutically effective dose. In still other embodiments of the bilayer tablet of the present disclosure, both ranolazine (preferably as the sustained release formulation) and dronedarone as the spray-dried phosphoric acid salt formulation are present in less than standard therapeutic doses. The expression “synergistically therapeutic amounts of dronedarone and ranolazine or pharmaceutically acceptable salt or salts thereof” is intended to encompass all possible combinations of standard and less than standard therapeutic doses of ranolazine, preferably, as the sustained release formulation and dronedarone as the spray-dried phosphoric acid salt formulation. Thus, one aspect of the present disclosure is to provide a method of treating atrial fibrillation or atrial flutter comprising administering a therapeutically effective amount of a bilayer tablet comprising a solid pharmaceutical composition of ranolazine as a sustained release formulation and a spray-dried phosphoric acid salt formulation of dronedarone as described herein

In another aspect, the present disclosure provides a solid pharmaceutical composition comprising sustained release formulation of ranolazine and spray-dried phosphoric acid salt formulation of dronedarone in a fixed dose combination as active pharmaceutical agents, and a pharmaceutically acceptable carrier. In a preferred embodiment, said pharmaceutical composition is a bilayer tablet comprising a first layer of ranolazine (preferably, sustained release formulation) and a second layer of spray-dried phosphoric acid salt formulation of dronedarone. Thus, it is also an object of the disclosure to provide a solid composition wherein the ranolazine is in the amount of from about 200 mg to about 1500 mg, preferably from about 375 mg to about 1000 mg; and the dronedarone phosphoric acid salt is in the amount of from about 50 mg to about 400 mg dronedarone equivalent. Preferably, the dose of spray-dried dronedarone is from about 50 mg to about 250 mg dronedarone equivalents and more preferably from about 75 mg to about 225 mg dronedarone equivalents. It is also an object of the present disclosure to provide a solid pharmaceutical composition in a bilayer tablet form comprising ranolazine, preferably as the sustained release formulation, at about 375 mg, about 500 mg, about 750 mg or about 1000 mg; the spray-dried dronedarone phosphoric acid salt formulation preferably in the amount of about 50 mg, about 75 mg, about 100 mg, about 112 mg, about 150 mg, or about 225 mg dronedarone equivalent; and a pharmaceutically acceptable carrier. A qualified care giver is in the best position to determine the appropriate dose or dosing regimen for a given patient. The qualified care giver will take into consideration such factors as the dose strength prescribed, age, weight, gender, patient history, presenting symptoms and their severity, co-presenting symptoms or diseases, frequency of administration, concomitant medications being taken by the patient, or whether a loading dose or a maintenance dose is required.

In one aspect, the present disclosure provides a method of treating atrial fibrillation comprising administering a therapeutically effective amount of one or more bilayer tablets further comprising ranolazine and one or more pharmaceutically acceptable excipients in a first layer, and a spray-dried phosphoric acid salt formulation of dronedarone further comprising HPMC E3 or HPMC E5 and one or more pharmaceutically acceptable excipients in a second layer.

In another aspect, the present invention provides a method of treating atrial flutter comprising administering a therapeutically effective amount of one or more bilayer tablets comprising ranolazine and one or more pharmaceutically acceptable excipients in a first layer, and a spray-dried phosphoric acid salt formulation of dronedarone further comprising HPMC E3 or HPMC E5 and one or more pharmaceutically acceptable excipients in a second layer.

### Active Ingredients and Compositions

### Ranolazine

Methods of preparing ranolazine are known to one of ordinary skill in the art. For example, sustained release formulation of ranolazine is disclosed in U.S. Pat. Nos. 6,503,911, 6,617,328, 6,303,607, 6,369,062, 6,525,057, 6,562,826, 6,620,814, 6852724, and 6864258. A particularly preferred method of preparing sustained release formulation of ranolazine is disclosed in U.S. Pat. No. 6,503,911, and international counterparts thereof, the entirety of which is incorporated herein by reference.

### Dronedarone

Methods of preparing dronedarone drug substance (base form) are known to one of skill in the art. For examples, U.S. Pat. No. 5,223,510 (the entirety of which is incorporated herein by reference) discloses dronedarone, N-(2-Butyl-3-(p-(3-(dibutylamino)propoxy)benzoyl)-5-benzofuranyl)methanesulfonamide, its pharmaceutically acceptable salts, and their use in the treatment of angina pectoris, hypertension, arrhythmias, and cerebral circulatory inefficiency.

### EXAMPLES

Dronedarone as used in this disclosure is well known in the art and may be prepared by following any one of many processes known to one of skill in the art including as disclosed in U.S. Pat. No. 5,223,510. Ranolazine may be prepared by conventional methods such as in the manner disclosed in U.S. Pat. No. 4,567,264, the entire disclosure of which is hereby incorporated by reference. Additionally, the abbreviations used throughout have the following meanings:
μM=micromolar
cm=centimeter
kg=kilogram
mA=milliamp
min=minute
mm=millimeter
mM=millimolar
ms=millisecond
MΩ=Mega Ohms

### Example 1

### Manufacturing Procedures

To manufacture the solid dispersion (formulation) of dronedarone as the spray-dried phosphoric acid salt formulation, the equipment train includes glass reactors, a spray dryer (Mobile Minor, GEA Niro, Søborg, Denmark) equipped with a two-fluid spray nozzle (1.0 mm orifice), and a tray-drying vacuum oven.

### Feed Solution Preparation

One batch of dronedarone feed solution at 15.0% (w/w) solid content was manufactured at a scale of 62.8 kg solution, corresponding to 9.42 kg of spray-dried powder. Two glass reactors were used to prepare the drug solution and the polymer solution separately. To prepare the drug solution, dronedarone drug substance was dispersed in diluted phosphoric acid solution, and gradually dissolved as a result of its reaction with phosphoric acid. 95% of the theoretical amount of phosphoric acid was initially charged to prepare the feed solution. After drug solution was solubilized, the remaining phosphoric acid solution was used to adjust the pH of the drug solution to 4.0±0.4. It is notable that the pH (at about room temperature) of the drug solution is measured with a pH probe (e.g. Double Junction Reference Electrode, Part No. (E16M321), Manufacturer: Radiometer Analytical) designed to measure pH of samples sensitive to chloride ion, since this particular solution is incompatible with conventional pH probes. To prepare the polymer solution, HPMC E3 LV or HPMC-E5 LV powder was dispersed in water, and gradually dissolved under gentle mixing. The drug solution and the polymer solution were then mixed to prepare the feed solution for spray drying.

### Spray Drying

The dronedarone feed solution was spray-dried using a closed-loop configuration. The supply fan was operated at 100% capacity to recirculate nitrogen as the drying gas at approximately 104 kg/hr. The condenser temperature was set at about 4° C. to remove the water from the recirculating nitrogen drying gas. The feed solution was sprayed at about 1.0 kg/hr. A 1.0 mm two-fluid spray nozzle was used for atomization. Nitrogen gas was also used as the atomization gas at about 2.0 bar atomization pressure. Under these processing conditions, the atomization ratio (ratio between atomization gas flow rate and feed solution spray rate) is about 3.0. The inlet temperature was maintained between about 84° C. and about 106° C. to keep the outlet temperature between about 55° C. and about 67° C. Prior to the initiation of spray drying, the system was equilibrated to the target condition by spraying pure water at a feed rate of about 0.85 kg/hr. The dronedarone feed solution was processed at about 1.0 kg/hr after the system reached equilibration. An in-line coarse filter, a ¾ inch PTFE TC screen gasket with 100 mesh stainless screen was used to filter any particulates in the feed solution. The filter was positioned after the glass reactor and before the peristaltic pump.

### Secondary Drying

Upon completion of the spray-drying process, dronedarone solid dispersion collected from the spray dryer was further dried in a nitrogen-purged tray-drying oven at about 40° C. under 1.0 bar vacuum until the residual water content was below 3%. The in-process water content was determined with Karl Fischer titration (KF).

### Composition of Final Feed Solution and Bulk Powder

Table 1 below describes the final composition of the feed solution and spray-dried bulk powder.

TABLE 1
Composition of Spray-Dried Dispersion
of Dronedarone Phosphoric Acid Salt

| | | Feed | Bulk |
|---|---|---|---|
| | Quality | Solution | Powder |
| Ingredient | Standard | Weight % | Weight % |
| Dronedarone | — | 9.57 | 63.8 |
| Phosphoric Acid ^{a} | NF | 1.68 | 11.2 |
| Hypromellose | NF | 3.75 | 25.0 |
| (HPMC) E3 LV | | | |
| Purified Water ^{b} | USP | 85.0 | — |
| Total | — | 100 | 100 |

^{a} Phosphoric acid NF is a mixture of phosphoric acid and water. The material contains not less than 85.0% H₃PO₄ and not more than 88.0% H₃PO₄. Percent w/w in this table represents phosphoric acid on a dry basis. The water from phosphoric acid is removed during the manufacturing process. At 1 to 1 molar ratio, dronedarone and H₃PO₄ react to form an in-situ water-soluble salt. The final pH of the feed solution is in the range of 4.0 ± 0.4.
^{b} The purified water for the preparation of feed solution for spray drying is removed during the manufacturing processes.

### Example 2

### Methods

The feed solutions for laboratory experiments were made by: 1) preparing an aqueous solution of the counter ion (e.g. phosphate, citrate, acetate); 2) adding dronedarone to the acid solution from the previous step; 3) separately preparing polymer (e.g. HPMC E3 LV, HPMC E5 LV, PVP, PVPVA, or HPMC AS) solution in water; and (4) combining solutions from Step (2) and (3). Total solid content of the feed solutions ranged from approximately 10% to 20% w/w. Optionally, the feed solution may be prepared by stepwise addition of the ingredients (phosphoric acid, dronedarone, and polymer) to the chosen solvent.

### Spray Drying of the Feed Solution:

The dronedarone feed solution was spray-dried using Buchi Mini Spray Dryer B-290 in a closed loop configuration. Compressed nitrogen is used as both the drying and atomization gas. The drying gas fan was operated at 100% capacity. The condenser temperature was operated at about 4° C. to remove the water from the recirculating drying gas. The feed solution was sprayed at approximately 3 g per minute. The atomization gas was set at about 70% of capacity. Inlet temperature was generally set at about 150° C. in order to maintain the outlet temperature between about 70° C. and about 80° C.

Physical stability was evaluated at several storage conditions, namely 40° C./75% RH and 25° C./60% RH in controlled temperature & relative humidity chambers. Solids were characterized either by visual inspection or by X-Ray Powder Diffraction when appropriate. Dronedarone solid dispersions have a very clear failure mechanism when exposed to high relative humidity. The solids transform from a white powder to a yellow, congealed, sticky mass.

### Spray Drying Trials

Table 2 below describes the results of experiments conducted resulting in the discovery of a stable solid spay-dried phosphoric acid salt formulation of dronedarone (dron).

TABLE 2
Suitability/Stability of Dronedarone Spray Drying Formulations
Utilizing Various Counter Ions and Polymers

| | Weight % | | |
|---|---|---|---|
| Mole ratio ( ) of | ratio ( ) of | Spray | |
| dronedarone base | dronedarone | Drying | Physical Stability |
| to counter ion | salt to polymer | Result* | (condition)** |
| Phosphate (1:2) | None | Fail | N/A |
| Phosphate (1:2) | PVP (1:1) | Success | No (40° C./75% RH) |
| | | | No (25° C./60% RH) |
| Phosphate (1:2) | PVPVA (1:1) | Success | No (40° C./75% RH) |
| | | | No (25° C./60% RH) |
| Phosphate (1:2) | HPMC AS (1:1) | Success | No (40° C./75% RH) |
| Phosphate (1:2) | HPMC E5 LV | Success | Yes (40° C./75% RH) |
| | (1:1) | | |
| Phosphate (1:1.15) | PVP (1:1) | Success | No (40° C./75% RH) |
| Phosphate (1:1.15) | PVPVA (1:1) | Success | No (40° C./75% RH) |
| Acetate (1:2) | None | Fail | N/A |
| Acetate (1:2) | PVP (1:1) | Success | No (40° C./75% RH) |
| Acetate (1:2) | PVPVA (1:1) | Success | No (40° C./75% RH) |
| | | | No (25° C./60% RH) |
| Acetate (1:2) | HPMC E5 LV | Success | No (40° C./75% RH) |
| | (1:1) | | No (25° C./60% RH) |
| Citrate (1:2) | HPMC E5 LV | Fail | N/A |
| | (1:1) | | |
| Phosphate (1:1) | HPMC E3 (3:1) | Success | Yes (40° C./75% RH) |
| Final Form | | | Yes (25° C./60% RH) |
| Phosphate (1:1) | HPMC E3 (6:1) | Success | Yes (40° C./75% RH) |
| | | | Yes (25° C./60% RH) |
| Phosphate (1:1) | HPMC E3 (9:1) | Success | Yes (40° C./75% RH) |
| | | | Yes (25° C./60% RH) |

*As used herein success means the product of the spray-drying was substantially a powder.
**Samples were stored under open conditions at the temperatures and relative humidities specified above.

Table 2 shows that the spray-dried phosphoric acid salt formulation of dronedarone formed by adding HPMC E3 or HPMC E5 provided solid, stable phosphoric acid salt formulation of dronedarone. The results further demonstrate that HPMC E3 and HPMC E5 produce stable spray-dried salts only with phosphoric acid.

### Composition of Spray Drying Trials

Table 3 below describes the composition of the feed solution used during spray drying trials.

TABLE 3
Spray Drying Feed Solution Compositions

| | | | | Counter | | | |
|---|---|---|---|---|---|---|---|
| | Mole ratio | | Dronedarone | ion | Polymer | | |
| | dronedarone:counter | | conc | conc | conc | Water | Organic |
| Counter ion | ion | polymer | (% w/w) | (% w/w) | (% w/w) | (% w/w) | (% w/w) |
| Phosphate | 1:2 | — | 10.0 | 3.0 | 0 | 87.0 | 0 |
| Phosphate | 1:2 | PVP | 10.0 | 3.0 | 10.0 | 77.0 | 0 |
| Phosphate | 1:2 | PVPVA | 5.0 | 1.5 | 5.0 | 88.5 | 0 |
| Phosphate | 1:2 | HPMC AS | 5.0 | 1.5 | 5.0 | 44.3 | 44.3 |
| | | | | | | | Acetone |
| Phosphate | 1:2 | HPMC E5 | 10.0 | 3.0 | 10.0 | 77.0 | 0 |
| | | LV | | | | | |
| Phosphate | 1:1.15 | PVP | 10.0 | 1.7 | 10.0 | 78.3 | 0 |
| Phosphate | 1:1.15 | PVPVA | 10.0 | 1.7 | 10.0 | 78.3 | 0 |
| Acetate | 1:2 | — | 10.0 | 3.0 | 10.0 | 77.0 | 0 |
| Acetate | 1:2 | PVP | 5.0 | 1.5 | 5.0 | 88.5 | 0 |
| Acetate | 1:2 | PVPA | 10.0 | 3.0 | 10.0 | 77.0 | 0 |
| Acetate | 1:2 | HPMC E5 | 5.0 | 1.5 | 5.0 | 88.5 | 0 |
| | | LV | | | | | |
| Citrate | 1:2 | HPMC E5 | 2.0 | 0.6 | 2.0 | 82.1 | 13.3 |
| | | LV | | | | | Acetonitrile |
| Phosphate | 1:1 | HPMC E3 | 9.6 | 1.68 | 3.75 | 85.0 | 0 |
| | | LV | | | | | |

### Example 2A

### Methods

Additional laboratory experiments were conducted to explore various feed solution compositions. The feed solutions for laboratory experiments were made by: (1) preparing phosphoric acid solution in water and/or solvent; (2) dry blending dronedarone and polymer (HPMC E3); (3) Slowly adding the dry blend to the phosphoric acid solution from step (1); Total solid content of the feed solutions ranged from approximately 15% to 30% w/w. Optionally, the feed solution may be prepared by stepwise addition of the ingredients (phosphoric acid, dronedarone, and polymer) to the chosen solvent.

### Spray Drying of the Feed Solution:

The dronedarone feed solution was spray-dried using Buchi® Mini Spray Dryer B-290 (Buchi Corporation) in a closed or opened loop configuration. Compressed nitrogen is used as both the drying and atomization gas. The drying gas fan was operated at 100% capacity. The condenser temperature was operated at about 4° C. to 10° C. to remove the water from the recirculating drying gas. The feed solution was sprayed at a rate from 2 to 7 g per minute. The atomization gas was set at about 70% of capacity. Inlet temperature was generally set between 100° C. and 160° C. in order to maintain the outlet temperature between about 60° C. and about 85° C.

Physical stability was evaluated at several storage conditions, namely 40° C./75% RH and 25° C./60% RH in controlled temperature & relative humidity chambers. Solids were characterized either by visual inspection or by X-Ray Powder Diffraction when appropriate. In all cases described below, the resulting spray-dried dronedarone dispersions were physically stable.

### Composition of Feed Solution

For all trials described below, the concentration of polymer (HPMC E3) was fixed at 25% of the solids content. The overall solids content, mole ratio of phosphouric acid counterion to dronedarone, cosolvent used, and weight ratio of water to cosolvent were varied between trials. Table 4 below describes the composition of the feed solution used during spray drying trials.

TABLE 4
Spray Drying Feed Solution Compositions
with Various Co-Solvents and Ratios

| Solids | | Counterion Mole | | Weight |
|---|---|---|---|---|
| Content | Dronedarone | Ratio (mol Phos- | | Ratio (g Co- |
| in Feed | conc | phouric Acid:mol | | solvent:g |
| (% w/w) | (% w/w) | Dronedarone) | Cosolvent | Water |
| 15 | 9.6 | 1:1 | None | NA |
| 15 | 9.7 | 0.97:1.03 | None | NA |
| 15 | 9.4 | 1.05:0.95 | None | NA |
| 20 | 12.8 | 1:1 | None | NA |
| 25 | 15.9 | 1:1 | None | NA |
| 15 | 9.6 | 1:1 | Acetone | 20:80 |
| 15 | 9.6 | 1:1 | Acetone | 50:50 |
| 25 | 15.9 | 1:1 | Acetone | 20:80 |
| 30 | 19.1 | 1:1 | Acetone | 20:80 |
| 25 | 15.9 | 1:1 | Acetone | 50:50 |
| 30 | 19.1 | 1:1 | Acetone | 50:50 |
| 25 | 15.9 | 1:1 | Ethanol | 50:50 |
| 25 | 15.9 | 1:1 | Ethanol | 80:20 |

### Example 2B

### Manufacturing Procedures

To manufacture the solid dispersion (formulation) of dronedarone as the spray-dried phosphoric acid salt formulation, the equipment train includes glass reactors, a spray dryer (FSD 12.5, GEA Niro, Søborg, Denmark) equipped with a pressure nozzle, and a double cone dryer.

### Feed Solution Preparation

One batch of dronedarone feed solution at 20.0% (w/w) solid content was manufactured at a scale of 1040.9 kg solution, corresponding to 207.8 kg of spray-dried powder. To prepare the drug solution, water was charged to the reactor and the polymer (HPMC E3 LV) was added and gradually dissolved under gentle mixing. Next, 100% of the theoretical amount of phosphoric acid, based on the assay value of the phosphoric acid, was charged to the reactor, followed by ethanol to form a solution of the HPMC E3 LV polymer in an 80:20 w/w mixture of ethanol and water. The dronedarone drug substance was dispersed in this solution, and gradually dissolved as a result of its reaction with phosphoric acid.

### Spray Drying

The dronedarone feed solution was spray-dried using a closed-loop configuration. The supply fan was operated at 100% capacity to recirculate nitrogen as the drying gas at approximately 1500 kg/hr. The condenser temperature was set at about 0° C. to remove the water and ethanol from the recirculating nitrogen drying gas. The feed solution was sprayed at about 95 kg/hr. A 1.06 mm pressure spray nozzle was used for atomization. The inlet temperature was maintained between about 90° C. and about 130° C. to keep the outlet temperature between about 45° C. and about 55° C. Prior to the initiation of spray drying, the system was equilibrated to the target condition by spraying and 80:20 w/w mixture of ethanol and water at a feed rate of about 76 kg/hr. The dronedarone feed solution was processed at about 95 kg/hr after the system reached equilibration.

### Secondary Drying

Upon completion of the spray-drying process, dronedarone solid dispersion collected from the spray dryer was further dried in a nitrogen-purged bi-conical dryer at about 40° C. under 0.85 to 1.0 bar vacuum for 84 hours.

### Composition of Final Feed Solution and Bulk Powder

Table 5 below describes the final composition of the feed solution and spray-dried bulk powder.

TABLE 5
Composition of Spray-Dried Dispersion
of Dronedarone Phosphoric Acid Salt

| | | Feed | Bulk |
|---|---|---|---|
| | Quality | Solution | Powder |
| Ingredient | Standard | Weight % | Weight % |
| Dronedarone | — | 12.75 | 63.8 |
| Phosphoric Acid ^{a} | NF | 2.30 | 11.2 |
| Hypromellose | NF | 4.91 | 25.0 |
| (HPMC) E3 LV | | | |
| Purified Water ^{b} | USP | 15.96 | — |
| Ethanol ^{b} | USP | 64.08 | — |
| Total | — | 100 | 100 |

^{a} Phosphoric acid NF is a mixture of phosphoric acid and water. The material contains not less than 85.0% H₃PO₄ and not more than 88.0% H₃PO₄. Percent w/w in this table represents phosphoric acid on a dry basis. The water from phosphoric acid is removed during the manufacturing process. At 1 to 1 molar ratio, dronedarone and H₃PO₄ react to form an in-situ water-soluble salt.
^{b} The purified water and ethanol for the preparation of feed solution for spray drying is removed during the manufacturing processes.

### Example 3

### Process for Preparing Spray-Dried Dronedarone Phosphoric Acid Salt Tablet, 225 mg

The base form (free base) of dronedarone is converted in-situ to the phosphate salt and processed by aqueous spray drying, and the isolated solid spray-dried formulation (dispersion) of dronedarone is further processed with a conventional dry granulation. Good compressibility of the spray-dried material makes the formulation amenable to a dry granulation process. A roller compaction and dry granulation process may be used for the preparation of solid spray-dried phosphoric acid salt formulation of dronedarone solid dispersion tablets. Formulation blends are densified into granules with good flow and compaction properties for compression.

The spray-dried phosphoric acid salt formulation of dronedarone solid dispersion tablets are formulated as follows (Table 6):

TABLE 6
Composition of Dronedarone Solid Dispersion Tablets, 225 mg

| | | | |
|---|---|---|---|
| | | Amt/Tablet | |
| Component | Weight % | (mg) | Function |

Intragranular

| | | | |
|---|---|---|---|
| Dronedarone Solid Dispersion | 71.6 | 352.7 | Active |
| 63.8% w/w^{a} | | | Ingredient |
| Microcrystalline Cellulose | 9.9 | 48.9 | Diluent |
| (Avicel PH-101) | | | |
| Colloidal Silicon Dioxide | 1.1 | 5.4 | Glidant |
| (Aerosil 200) | | | |
| Magnesium Stearate | 1.7 | 8.1 | Lubricant |
| (HyQual) | | | |

Extragranular

| | | | |
|---|---|---|---|
| Microcrystalline Cellulose | 3.00 | 14. | Diluent |
| (Avicel PH-102) | | | |
| Crospovidone | 12.0 | 59.1 | Disintegrant |
| (Kollidon CL) | | | |
| Magnesium Stearate | 0.8 | 3.7 | Lubricant |
| (HyQual) | | | |
| Total for Core Tablet | 100.0 | 492.7 | — |

Film-coating

| | | | |
|---|---|---|---|
| Opadry II White | — | 14.9^{d} | Film-Coat |
| 85F18422^{b} | | | |
| Purified Water^{c} | — | — | Processing Aid |
| Total for Coated Tablets | — | 507.5 | |

^{a}The composition of the solid dispersion is shown in Table 1. During the manufacturing of dronedarone solid dispersion tablets, the actual quantity of dronedarone solid dispersion, 63.8% w/w, was adjusted based on the drug content factor with concomitant adjustment on the quantity of microcrystalline cellulose. Additionally, the dronedarone solid dispersion can be prepared by any of the processes described in Examples 1, 2, 2A, or 2B.
^{b}Opadry II White 85F18422 was prepared as a 15% w/w aqueous suspension for film coating. Opadry II White 85F18422 contains 40.0% polyvinyl alcohol USP, 20.2% w/w polyethylene glycol 3350 NF, 25.0% w/w titanium dioxide USP, and 14.8% w/w talc USP.
^{c}sufficient water is used for film coating and is removed during the coating process.
^{d}Represents theoretical weight gain of 3% (range of 2 to 4%).

### Manufacturing Process for Dronedarone Solid Dispersion Tablets, 225 mg

1. Blend all intragranular components except for magnesium stearate in a V shell blender for 6 minutes at 25 rpm
2. Pass the blend through a Comill fitted with a screen with 0.055 inch round opening
3. Charge intragranular magnesium stearate into the V shell blender and blend for 2 minutes at 25 rpm
4. Dry granulate the lubricated blend with a Gerteis roller compactor
5. Blend the roller-compacted granules with the extragranular excipients except for magnesium stearate in a V shell blender for 6 minutes at 25 rpm
6. Charge extragranular magnesium stearate into the V shell blender and blend for 2 minutes at 25 rpm
7. Tableting the final blend using a rotary tablet press with 7/16 inch round, standard concave, tablet tooling; the target hardness is 15 kp
8. Coat the tablets using a perforated tablet coater.

### Example 4

The composition of dronedarone solid dispersion tablets, 75 mg, is presented in Table 7. Except for the tooling for tabletting and the target hardness, the manufacturing process is essentially the same as what is used to manufacture the solid dispersion tablets, 225 mg, as described in Example 3.

The tooling for the compression of dronedarone solid dispersion tablets, 75 mg, is 11/32 inch round, standard concave tablet tooling; the target hardness is 9 kp.

TABLE 7
Composition of Dronedarone Solid Dispersion Tablets, 75 mg

| | | | |
|---|---|---|---|
| | | Amt/Tablet | |
| Component | Weight % | (mg) | Function |

Intragranular

| | | | |
|---|---|---|---|
| Dronedarone Solid Dispersion | 45.0 | 117.6 | Active |
| 63.8% w/w^{a} | | | Ingredient |
| Microcrystalline Cellulose | 20.8 | 54.2 | Diluent |
| (Avicel PH-105) | | | |
| Crospovidone | 15.0 | 39.2 | Disintegrant |
| (Kollidon CL) | | | |
| Colloidal Silicon Dioxide | 2.0 | 5.2 | Glidant |
| (Aerosil 200) | | | |
| Magnesium Stearate | 1.5 | 3.9 | Lubricant |
| (HyQual) | | | |

Extragranular

| | | | |
|---|---|---|---|
| Microcrystalline Cellulose | 10.0 | 26.1 | Diluent |
| (Avicel PH-102) | | | |
| Crospovidone | 5.0 | 13.1 | Disintegrant |
| (Kollidon CL) | | | |
| Magnesium Stearate | 0.8 | 2.0 | Lubricant |
| (HyQual) | | | |
| Total for Core Tablet | 100.0 | 261.3 | — |

Film-coating

| | | | |
|---|---|---|---|
| Opadry II White | — | 7.8^{d} | Film-Coat |
| 85F18422^{b} | | | |
| Purified Water^{c} | — | — | Processing Aid |
| Total for Coated Tablets | — | 269.2 | |

^{a}The composition of the solid dispersion is shown in Table 1. During the manufacturing of dronedarone solid dispersion tablets, the actual quantity of dronedarone solid dispersion, 63.8% w/w, was adjusted based on the drug content factor with concomitant adjustment on the quantity of microcrystalline cellulose. Additionally, the dronedarone solid dispersion can be prepared by any of the processes described in Examples 1, 2, 2A, or 2B.
^{b}Opadry II White 85F18422 was prepared as a 15% w/w aqueous suspension for film coating. Opadry II White 85F18422 contains 40.0% polyvinyl alcohol USP, 20.2% w/w polyethylene glycol 3350 NF, 25.0% w/w titanium dioxide USP, and 14.8% w/w talc USP.
^{c}Sufficient water is used for film coating and is removed during the coating process.
^{d}Represents theoretical weight gain of 3% (range of 2 to 4%)

### Example 5

### Ranolazine (600 mg) and Dronedarone (225 mg) Fixed Dose Combination (FDC) Bilayer Tablets

TABLE 8
Ranolazine Layer (600 mg)

| | | | |
|---|---|---|---|
| | | Amt/layer | |
| Component | Weight % | (mg) | Function |

Intragranular

| | | | |
|---|---|---|---|
| Ranolazine, API | 75.0 | 600.0 | Active |
| | | | Ingredient |
| Microcrystalline Cellulose | 10.6 | 84.8 | Diluent |
| (Avicel PH-101) | | | |
| Methacrylic acid-Ethyl | 10.0 | 80.0 | Drug Release |
| Acrylate Copolymer (1:1), | | | Retardant |
| Type A | | | |
| (Eudragit L-100) | | | |
| Hypromellose 2910 | 2.0 | 16.0 | Drug Release |
| (Methocel E5 LV) | | | Retardant |
| Sodium Hydroxide | 0.4 | 3.2 | Neutralizing |
| | | | Agent |
| Purified Water^{a} | — | | Granulation |
| | | | Medium |

Extragranular

| | | | |
|---|---|---|---|
| Magnesium Stearate | 2.0 | 16.0 | Lubricant |
| (HyQual) | | | |
| Total for Ranolazine Layer | 100.0 | 800.0 | — |

^{a}Sufficient water is used for high-shear wet granulation and is removed during the fluid bed drying process.

TABLE 9
Dronedarone Layer (225 mg)

| | | | |
|---|---|---|---|
| | | Amt/layer | |
| Component | Weight % | (mg) | Function |

Intragranular

| | | | |
|---|---|---|---|
| Dronedarone Solid Dispersion | 71.6 | 352.7 | Active |
| 63.8% w/w^{a} | | | Ingredient |
| Microcrystalline Cellulose | 9.9 | 48.9 | Diluent |
| (Avicel PH-101) | | | |
| Colloidal Silicon Dioxide | 1.1 | 5.4 | Glidant |
| (Aerosil 200) | | | |
| Magnesium Stearate | 1.7 | 8.1 | Lubricant |
| (HyQual) | | | |

Extragranular

| | | | |
|---|---|---|---|
| Microcrystalline Cellulose | 3.0 | 14.8 | Diluent |
| (Avicel PH-102) | | | |
| Crospovidone | 12.0 | 59.1 | Disintegrant |
| (Kollidon CL) | | | |
| Magnesium Stearate | 0.7 | 3.7 | Lubricant |
| (HyQual) | | | |
| Total for Dronedarone Layer | 100.0 | 492.7 | — |

^{a}The composition of the solid dispersion is shown in Table 1. During the manufacturing of dronedarone solid dispersion tablets, the actual quantity of dronedarone solid dispersion, 63.8% w/w, was adjusted based on the drug content factor with concomitant adjustment on the quantity of microcrystalline cellulose. Additionally, the dronedarone solid dispersion can be prepared by any of the processes described in Examples 1, 2, 2A, or 2B.

### Manufacturing Process for Ranolazine and Dronedarone Phosphoric Acid Salt Fixed Dose Combination (FDC) Tablets

### Ranolazine Granules

1. Blend Ranolazine, Hypromellose, microcrystalline cellulose and Eudragit L100-55 in a bin blender for 10 minutes
2. Transfer the blend to a high-shear granulator
3. Spray sodium hydroxide solution into the high shear mixer for granulation
4. Dry the wet granules using a fluid bed dryer; the fluid bed dryer until a target LOD of 2.0% is reached.
5. Mill and blend the dried granulation with magnesium stearate in a bin blender

### Dronedarone (Phosphoric Acid Salt Formulation) Granules

1. Blend all intragranular components except for magnesium stearate in a V shell blender for 6 minutes at 25 rpm
2. Pass the blend through a Comill fitted with a screen with 0.055 inch round opening
3. Charge intragranular magnesium stearate into the V shell blender and blend for 2 minutes at 25 rpm
4. Dry granulate the lubricated blend with a Gerteis roller compactor
5. Blend the roller-compacted granules with the extragranular excipients except for magnesium stearate in a V shell blender for 6 minutes at 25 rpm
6. Charge extragranular magnesium stearate into the V shell blender and blend for 2 minutes at 25 rpm

### Bilayer Tablets

Compress ranolazine granules and dronedarone (phosphoric acid salt formulation) granules using a bilayer tablet press; ranolazine is in the first layer and dronedarone is in the second layer.

### Example 6

### Ranolazine (375 mg) and Dronedarone (112.5 mg) Fixed Dose Combination (FDC) Bilayer Tablets

The composition of ranolazine (375 mg) and dronedarone (112.5 mg) fixed dose combination tablet are presented in Tables 10 and 11 respectively. The manufacturing process is the same as what is used to manufacture the fixed dose combination tablets described in Example 5.

TABLE 10
Ranolazine Layer (375 mg)

| | | | |
|---|---|---|---|
| | | Amt/layer | |
| Component | Weight % | (mg) | Function |

Intragranular

| | | | |
|---|---|---|---|
| Ranolazine, API | 75.0 | 375.0 | Active |
| | | | Ingredient |
| Microcrystalline Cellulose | 10.6 | 53.0 | Diluent |
| (Avicel PH-101) | | | |
| Methacrylic acid-Ethyl | 10.0 | 50.0 | Drug Release |
| Acrylate Copolymer (1:1), | | | Retardant |
| Type A | | | |
| (Eudragit L-100) | | | |
| Hypromellose 2910 | 2.0 | 10.0 | Drug Release |
| (Methocel E5 LV) | | | Retardant |
| Sodium Hydroxide | 0.4 | 2.0 | Neutralizing |
| | | | Agent |
| Purified Water^{a} | — | | Granulation |
| | | | Medium |

Extragranular

| | | | |
|---|---|---|---|
| Microcrystalline Cellulose | 0.5 | 2.5 | Diluent |
| (Avicel PH-101) | | | |
| Colloidal SiO2 | 0.5 | 2.5 | Glident |
| (Aerosil 200) | | | |
| Magnesium Stearate | 2.0 | 10.0 | Lubricant |
| (HyQual) | | | |
| Total for Ranolazine Layer | 100.0 | 500.0 | — |

^{a}Sufficient water is used for high-shear wet granulation and is removed during the fluid bed drying process.

TABLE 11
Dronedarone Layer (112.5 mg)

| | | | |
|---|---|---|---|
| | | Amt/layer | |
| Component | Weight % | (mg) | Function |

Intragranular

| | | | |
|---|---|---|---|
| Dronedarone Solid Dispersion | 35.3 | 176.3 | Active |
| 63.8% w/w^{a} | | | Ingredient |
| Microcrystalline Cellulose | 31.0 | 154.9 | Diluent |
| (Avicel PH-105) | | | |
| Crospovidone | 15.0 | 75.0 | Disintegrant |
| (Kollidon CL) | | | |
| Colloidal Silicon Dioxide | 2.0 | 10.0 | Glidant |
| (Aerosil 200) | | | |
| Magnesium Stearate | 1.0 | 5.0 | Lubricant |
| (HyQual) | | | |

Extragranular

| | | | |
|---|---|---|---|
| Microcrystalline Cellulose | 10.0 | 50.0 | Diluent |
| (Avicel PH-102) | | | |
| Crospovidone | 5.00 | 25.0 | Disintegrant |
| (Kollidon CL) | | | |
| Magnesium Stearate | 0.8 | 3.8 | Lubricant |
| (HyQual) | | | |
| Total for Dronedarone Layer | 100.0 | 500.0 | — |

^{a}The composition of the solid dispersion is shown in Table 1. During the manufacturing of dronedarone solid dispersion tablets, the actual quantity of dronedarone solid dispersion, 63.8% w/w, was adjusted based on the drug content factor with concomitant adjustment on the quantity of microcrystalline cellulose. Additionally, the dronedarone solid dispersion can be prepared by any of the processes described in Examples 1, 2, 2A, or 2B.

### Example 7

### Ranolazine (375 mg) and Dronedarone (225 mg) Fixed Dose Combination (FDC) Bilayer Tablets

It is also possible to prepare a FDC bilayer tablet without roller compaction of the dronedarone phosphate salt spray-dried dispersion. The composition of ranolazine (375 mg) and dronedarone (225 mg) fixed dose combination tablet is presented in Tables 12 and 13 respectively.

TABLE 12
Ranolazine Layer (375 mg)

| | | | |
|---|---|---|---|
| | | Amt/layer | |
| Component | Weight % | (mg) | Function |

Intragranular

| | | | |
|---|---|---|---|
| Ranolazine, API | 75.0 | 375.0 | Active |
| | | | Ingredient |
| Microcrystalline Cellulose | 10.6 | 53.0 | Diluent |
| (Avicel PH-101) | | | |
| Methacrylic acid-Ethyl | 10.0 | 50.0 | Drug Release |
| Acrylate Copolymer (1:1), | | | Retardant |
| Type A | | | |
| (Eudragit L-100) | | | |
| Hypromellose 2910 | 2.0 | 10.0 | Drug Release |
| (Methocel E5 LV) | | | Retardant |
| Sodium Hydroxide | 0.4 | 2.0 | Neutralizing |
| | | | Agent |
| Purified Water^{a} | — | | Granulation |
| | | | Medium |

Extragranular

| | | | |
|---|---|---|---|
| Microcrystalline Cellulose | 0.5 | 2.5 | Diluent |
| (Avicel PH-101) | | | |
| Colloidal SiO2 | 0.5 | 2.5 | Glident |
| (Aerosil 200) | | | |
| Magnesium Stearate | 2.0 | 10.0 | Lubricant |
| (HyQual) | | | |
| Total for Ranolazine Layer | 100.0 | 500.0 | — |

^{a}Sufficient water is used for high-shear wet granulation and is removed during the fluid bed drying process.

TABLE 13
Dronedarone Layer (225 mg)

| | | Amt/layer | |
|---|---|---|---|
| Component | Weight % | (mg) | Function |
| Dronedarone Solid Dispersion | 64.1 | 352.7 | Active |
| 63.8% w/w^{a} | | | Ingredient |
| Microcrystalline Cellulose | 9.7 | 53.3 | Diluent |
| (Avicel PH-102) | | | |
| Lactose Anhydrous (DCL-21) | 9.7 | 53.3 | Diluent |
| Crospovidone | 15.0 | 82.5 | Disintegrant |
| (Kollidon CL) | | | |
| Colloidal Silicon Dioxide | 0.75 | 4.1 | Glidant |
| (Aerosil 200) | | | |
| Magnesium Stearate | 0.75 | 4.1 | Lubricant |
| (HyQual) | | | |
| Total for Dronedarone Layer | 100.0 | 550.0 | — |

^{a}The composition of the solid dispersion is shown in Table 1. During the manufacturing of dronedarone solid dispersion tablets, the actual quantity of dronedarone solid dispersion, 63.8% w/w, was adjusted based on the drug content factor with concomitant adjustment on the quantity of microcrystalline cellulose. Additionally, the dronedarone solid dispersion can be prepared by any of the processes described in Examples 1, 2, 2A, or 2B.

### Process for Ranolazine and Dronedarone Phosphoric Acid Salt Fixed Dose Combination (FDC) Tablets

### Ranolazine Granules

1. Blend Ranolazine, Hypromellose, microcrystalline cellulose and Eudragit L100-55 in a bin blender for 10 minutes
2. Transfer the blend to a high-shear granulator
3. Spray sodium hydroxide solution into the high shear mixer for granulation
4. Dry the wet granules using a fluid bed dryer; the fluid bed dryer until a target LOD of 2.0% is reached.
5. Mill and blend the dried granulation with magnesium stearate in a bin blender

### Dronedarone (Phosphoric Acid Salt Formulation) Powder Blend

1. Blend dronedarone phosphoric acid salt spray-dried dispersion and other components except for magnesium stearate in a Turbula mixer for 10 minutes. Charge magnesium stearate into the Turbula mixer and blend for 10 minutes

### Bilayer Tablets

Compress ranolazine granules and dronedarone (phosphoric acid salt formulation) powder blend or granules using a Carver press or other process known to one of skill in the art; ranolazine is in the first layer and dronedarone is in the second layer.

We claim:

1-25. (canceled)

26. A process for making a bilayer tablet comprising ranolazine in a first layer and stable solid spray-dried phosphoric acid salt formulation of dronedarone in a second layer further comprising the steps of:
a. providing a powder blend of spray-dried phosphoric acid salt formulation of dronedarone with suitable excipients;
b. optionally processing the powder blend from step (a) into granules with suitable flow and compression properties;
c. providing a powder blend of ranolazine with suitable excipients;
d. processing the powder blend from step (c) with suitable excipients into granules with suitable flow and compression properties; and
e. forming a bilayer tablet by compressing granules from step (b) or powder blend from step (a) and the granules from step (d) using a bilayer tablet press, wherein the granules from step (d) are in a first layer and the granules from step (b) or powder blend from step (a) are in a second layer.

27. The process for making a bilayer tablet according to claim 26 further comprising the steps of:
a. providing a powder blend of spray-dried phosphoric acid salt formulation of dronedarone with suitable excipients;
b. processing the powder blend from step (a) into granules with suitable flow and compression properties;
c. providing a powder blend of ranolazine with suitable excipients;
d. processing the powder blend from step (c) with suitable excipients into granules with suitable flow and compression properties; and
e. forming a bilayer tablet by compressing granules from step (b) and the granules from step (d) using a bilayer tablet press.

28. The process according to claim 26 further comprising the steps of:
a. providing granules of spray-dried phosphoric acid formulation of dronedarone;
b. providing granules of ranolazine
c. forming a bilayer tablet by compressing dronedarone granules from step (a) and the ranolazine granules from step (b) using a bilayer tablet press, wherein the dronedarone and ranolazine granules are in separate layers.

29. The process according to claim 26 for making a stable solid spray-dried phosphoric acid salt formulation of dronedarone further comprising the steps of:
a. dissolving the base form of dronedarone in a solution of phosphoric acid to form a dronedarone solution;
b. optionally adjusting the pH of the dronedarone solution from step (a) to about 4.0 with additional phosphoric acid as necessary;
c. adding HPMC E3 or HPMC E5 to the dronedarone solution from step (b);
d. spray drying the dronedarone solution from step (c) to provide a solid comprising spray-dried phosphoric acid salt formulation of dronedarone; and
e. optionally drying the solid spray-died phosphoric acid salt formulation of dronedarone.

30. The process for making a stable solid spray-dried phosphoric acid salt formulation of dronedarone further comprising the steps of:
a. dissolving the base form of dronedarone in a solution of 1:1 molar equivalent of phosphoric acid (based on dronedarone base) to form a dronedarone solution;
b. adding HPMC E3 or HPMC E5 or solution thereof to the dronedarone solution from step (a);
c. spray drying the dronedarone solution from step (b) to achieve a solid spray-dried dronedarone phosphoric acid salt formulation; and
d. optionally drying the solid spray-dried phosphoric acid salt formulation of dronedarone.

31. The process according to claim 26 further comprising the steps of:
a. dissolving HPMC E3 or HPMC E5 and the base form of dronedarone in a suitable solvent or solvent mixture that contains 1:1 molar equivalent of phosphoric acid (based on dronedarone base) to form a dronedarone solution;
b. spray drying the dronedarone solution from step (a) to achieve a solid spray-dried dronedarone phosphoric acid salt formulation; and
c. optionally drying the solid spray-dried phosphoric acid salt formulation of dronedarone.

32. The process according to claim 29 wherein the weight % ratio of dronedarone base to HPMC E3 or HPMC E5 polymer is from about 0.5:1 to about 15:1.

33. The process according to claim 29 wherein the weight % ratio of dronedarone base to HPMC E3 or HPMC E5 polymer is from about 1:1 to about 10:1.

34. The process according claim 29 wherein the weight % ratio of dronedarone base to HPMC E3 or HPMC E5 polymer is from about 1:1 to about 6:1.

35. The process according to claim 29 wherein the weight % ratio of dronedarone base to HPMC E3 or HPMC E5 polymer is from about 1:1 to about 2:1.

36. The process of any one of claim 26 wherein the ranolazine formulation is the sustained release formulation of ranolazine.

37. (canceled)
